(19) United States
(12) Patent Application Publication
(10) Pub. No.: US 2026/0363966 A1
(43) Pub. Date: Nov. 19, 2026

(54) HYDROREACTIVE SOLID COMPOUND AND METHOD OF MAKING THE SAME

(71) Applicants: Ilya Gonorovsky, Hollywood, FL (US); Zhanna Feldsher, Studio City, CA (US)

(72) Inventors: Ilya Gonorovsky, Hollywood, FL (US); Zhanna Feldsher, Studio City, CA (US)

(21) Appl. No.: 19/211,030

(22) Filed: May 16, 2025

(51) Int. Cl. A61K 8/02; A61Q 9/02

(52) U.S. Cl. CPC A61K 8/0216; A61Q 9/02; A61K 2800/882

(57) ABSTRACT

A hydroreactive solid compound in a form of a tablet or a pill, when mixed with fluids, transforms into a shaving cream or gel suitable for personal grooming. This innovation presents a compact, water-activated alternative to conventional aerosol and non-aerosol shaving products. The shaving tablet is designed for single-use or multi-dose formats and may be stored in portable containers, including integrated razor handles or travel cases. Upon the addition of a small amount of water, the tablet produces a lather or gel that lubricates and protects the skin during shaving.

### FIELD OF INVENTION

The present invention relates to a solid material engineered to chemically or physically react upon contact with water, transforming into a different state like foam to be used as consumer products and method of forming the same.

### BRIEF DESCRIPTION OF THE BACKGROUND

Shaving is an age-old grooming practice with origins dating back thousands of years. In ancient Mesopotamia and Egypt (circa 3000 B C E), rudimentary razors made from bronze or flint were used alongside naturally available substances such as oils, animal fats, or water to soften facial hair. In ancient Greece and Rome, olive oil was commonly applied to lubricate the skin prior to shaving.

During the Middle Ages, shaving was largely handled by barbers and often involved the use of hot towels, soap, and straight razors. Shaving habits and tools fluctuated based on cultural and religious norms of the time. By the 19th century, shaving had become more refined with the introduction of traditional shaving soaps—hard pucks of soap that required a brush and bowl to produce lather. These soaps, used in barbershops and homes alike, offered excellent slickness and durability. The professional straight-razor shave with hot lather became a mainstay of male grooming.

A major shift occurred in the 20th century, particularly in 1919, when Barbasol introduced the first brushless shaving cream, eliminating the need for a shaving brush and making daily grooming faster and more accessible. In the 1940s and 1950s, the advent of aerosol shaving foam further simplified the shaving process. These innovations were followed in later decades by the introduction of shaving gels, sensitive-skin formulations, and non-pressurized options that catered to more specific skin needs.

In the 21st century, there has been a dual trend of returning to traditional wet shaving methods (e.g., using artisan soaps and safety razors) while also expanding into natural, organic, and luxury shaving products. These modem formulations often feature botanical extracts, essential oils, and are free from synthetic additives like parabens and sulfates.

Current shaving products generally fall into several categories. Traditional shaving soaps, requiring water and a brush to lather. Shaving creams, available in tubes or tubs, which may be brushless or require lathering. Shaving gels, typically clear and brush-free, used for more precise grooming. Aerosol foams, which provide quick application but may lack skin-conditioning benefits. Brushless creams, often designed for travel or convenience. Natural and organic creams, marketed for their skin-friendly formulations. Luxury/artisan products, often small-batch and scent-focused. Recent innovations have also explored water-activated powders that turn into shaving foam or gel upon contact with water. These products aim to reduce packaging bulk and improve portability.

However, a significant limitation persists in all the above-mentioned solutions: they rely heavily on the availability of water, require some form of container or pressurized packaging, and often occupy non-trivial space-factors that are suboptimal for military personnel, hikers, travelers, and individuals in water-scarce or high-mobility environments.

Accordingly, there remains an unmet need for compact, waterless, and lightweight shaving solutions that eliminate the need for bulky tubes, aerosols, or water-based activation, while still offering skin-safe, effective shaving performance under extreme or minimal-resource conditions.

### SUMMARY OF THE INVENTION

A hydroreactive solid compound in a form of a tablet or a pill, when mixed with fluids, transforms into a shaving cream or gel suitable for personal grooming. This innovation presents a compact, water-activated alternative to conventional aerosol and non-aerosol shaving products. The shaving tablet is designed for single-use or multi-dose formats and may be stored in portable containers, including integrated razor handles or travel cases. Upon the addition of a small amount of water, the tablet produces a lather or gel that lubricates and protects the skin during shaving.

To create a solid version of shaving cream or gel such as hard pill or tablet when water is added, a combination of solid ingredients and water-absorbing or swelling agents that activate upon hydration. This method requires ingredients that are solid at room temperature but will soften and create a gel-like consistency when mixed with water.

The method for manufacturing the hydroreactive shaving tablet configured to transform into a shaving cream or gel upon contact with fluid, is also disclosed. The method comprising the steps of melting one or more solid fats selected from the group consisting of shea butter, cocoa butter, mango butter, and functional equivalents thereof to form a liquid base, adding one or more emulsifiers selected from the group consisting of stearic acid, glyceryl stearate, and functional equivalents thereof to the melted fats and mixing until dissolved, and incorporating one or more gelling agents selected from the group consisting of carbomer, xanthan gum, hydroxyethylcellulose, and functional equivalents thereof along with one or more binding agents selected from the group consisting of kaolin clay, cornstarch, arrowroot powder, and functional equivalents thereof.

The method further includes the steps of blending in an effervescent system comprising sodium bicarbonate and further adding one or more surfactants selected from the group consisting of sodium lauryl sulfate, sodium cocoyl isethionate, and functional equivalents thereof to form a semi-solid homogeneous mixture and adding one or more moisturizers selected from the group consisting of glycerin, aloe vera extract, coconut oil, shea butter, and functional equivalents thereof. The method further includes the step of molding the mixture into a pill or tablet shape using silicone molds and optionally applying compression and allowing the molded composition to cool and solidify at ambient temperature or under refrigeration for a period sufficient to produce a solid hydroreactive shaving tablet.

An advantage of the present invention is to provide a hydroreactive solid compound that is eco-friendly, thereby eliminating the need for aerosol propellants and significantly reduces plastic and metal waste associated with conventional shaving products.

Another advantage of the present invention is to provide a hydroreactive solid compound that is travel-friendly, compact, lightweight, and TSA-compliant, because tablets or pills can be easily stored in minimal space, such as in a razor handle or compact travel container.

Still another advantage of the present invention is to provide a hydroreactive solid compound that is customizable and can be formulated for various skin types, including sensitive skin, with optional natural, hypoallergenic, or fragrance-free ingredients.

The objects and advantages of the present invention will be more readily apparent from inspection of the following specification, taken in connection with the accompanying drawing, wherein like numerals refer to like parts throughout and in which an embodiment of the present invention is described and illustrated.

The exact manner in which the foregoing and other objects and advantages of the invention are achieved in practice will become more clearly apparent when reference is made to the following detailed description of the preferred embodiments of the invention described in detail in the following specification, where in like reference numbers indicate corresponding parts throughout.

### BRIEF DESCRIPTION OF THE DRAWINGS

Other advantages of the present invention will be readily appreciated as the same becomes better understood by reference to the following detailed description when considered in connection with the accompanying drawings wherein:

FIG. 1 is a perspective view of a single hydroreactive shaving tablet in a disc-shaped form, showing its compact structure;

FIG. 2 is a cross-sectional view of the shaving tablet of FIG. 1, illustrating the homogeneous distribution of ingredients throughout the solid matrix;

FIG. 3 is a schematic flow diagram of the manufacturing process, showing the sequential steps of melting, mixing, molding, and drying the hydroreactive composition;

FIG. 4 is a perspective view of a compact storage container holding multiple shaving tablets for travel use;

FIG. 5 is a diagram showing the hydration of the tablet upon the addition of water, resulting in transformation into a gel or cream; and

FIG. 6 is an exploded view of a shaving razor with an integrated storage compartment in the handle, designed to hold one or more tablets.

### DETAILED DESCRIPTION OF THE INVENTION

For purposes of this patent document, the terms “or” and “and” shall mean “and/or” unless stated otherwise or clearly intended otherwise by the context of their use. The term “a” shall mean “one or more” unless stated otherwise or where the use of “one or more” is clearly inappropriate. The terms “comprise,” “comprising,” “include,” and “including” are interchangeable and not intended to be limiting. For example, the term “including” shall be interpreted to mean “including, but not limited to.”

Additionally, as used herein, the singular forms “a,” “an,” and “the” include plural referents unless the context clearly dictates otherwise. Thus, for example, the term “module” is intended to mean one or more modules or a combination of modules. Furthermore, as used herein, the term “based on” includes based at least in part on. Thus, a feature that is described as based on some cause, can be based only on that cause, or based on that cause and on one or more other causes.

It will be apparent that multiple embodiments of this disclosure may be practiced without some or all of these specific details. In other instances, well-known process operations have not been described in detail in order not to unnecessarily obscure the present embodiments. The following description of embodiments includes references to the accompanying drawing. The embodiments can be combined, other embodiments can be utilized, or structural, logical and operational changes can be made without departing from the scope of what is claimed. The following detailed description is, therefore, not to be taken in a limiting sense, and the scope is defined by the appended claims and their equivalents.

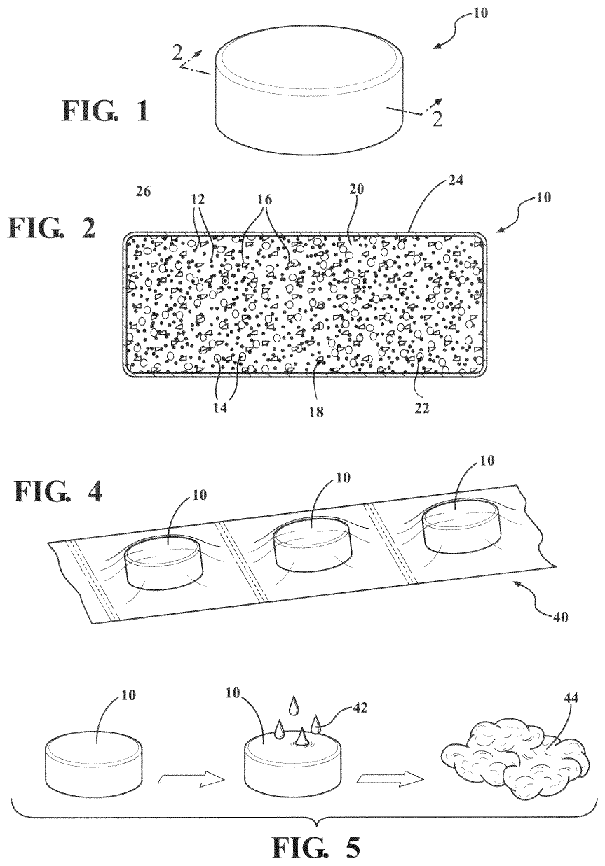

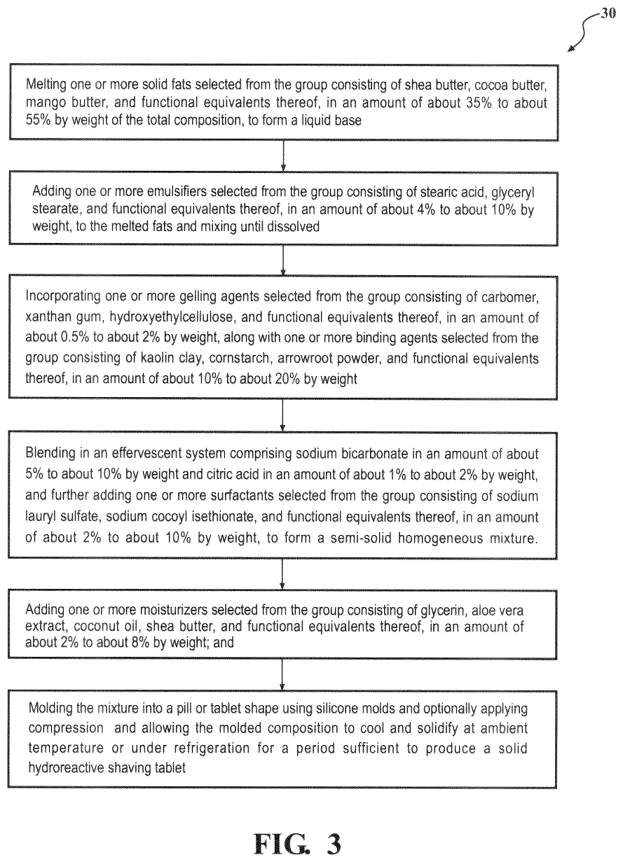

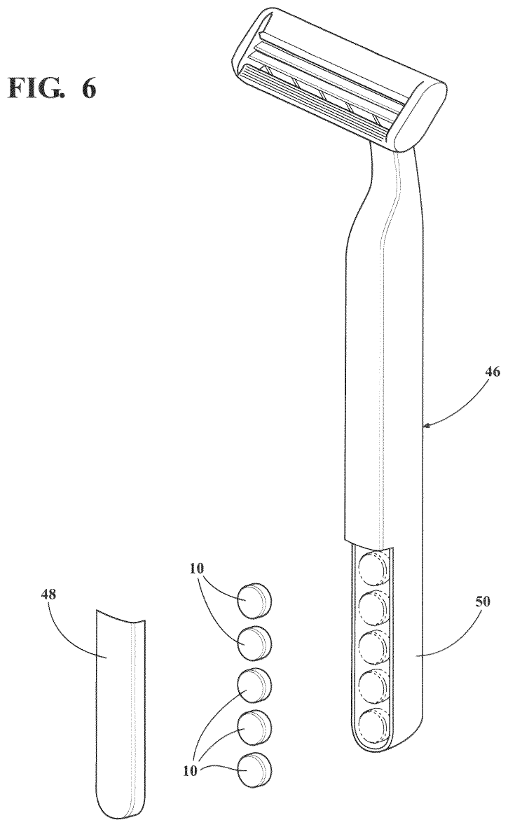

FIG. 1 is a perspective view of a single hydroreactive shaving tablet in a disc-shaped form, showing its compact structure, as generally shown at 10. FIG. 2 is a cross-sectional view of the shaving tablet 10 of FIG. 1, illustrating the homogeneous distribution of ingredients 12 through 26 throughout the solid matrix of the tablet 10. FIG. 3 is a schematic flow diagram of the manufacturing process, generally indicated at 30, showing the sequential steps of melting, mixing, molding, and drying the hydroreactive composition. FIG. 4 is a perspective view of a compact storage containers, generally shown at 40, holding multiple shaving tablets 10 for travel use. FIG. 5 is a diagram showing the hydration of the tablet 10 upon the addition of water, generally indicated at 42, resulting in transformation into a gel or cream, generally indicated at 44. FIG. 6 is an exploded view of a shaving razor 46 with an integrated storage compartment 48 in a handle 50 of the razor 46, designed to hold one or more tablets 10.

Alluding to the above, the hydroreactive solid compound in the form of the tablet or the pill 10 that, upon contact with water, transforms into a shaving cream or gel. This innovation provides a portable, eco-friendly, and water-activated alternative to traditional aerosol and non-aerosol shaving products. The composition consists of a blend of functional ingredients, each contributing to the formation, performance, stability, and sensory characteristics of the final product.

Each component is described below, along with its function, suitable percentage range by weight (w/w), and possible equivalents. Solid fats 12 form the structural base of the tablet and provide emollient properties, helping to nourish and protect the skin during shaving. The preferred concentration of solid fats in the final formulation is approximately 35% to 55% by weight. Suitable fats include shea butter, cocoa butter, and mango butter. These ingredients may be used individually or in combination.

The hydroreactive solid compound includes equivalent solid fats with similar melting profiles and emollient properties may include kokum butter, sal butter, or illipe butter. Emulsifiers 14 serve to stabilize the blend of hydrophilic and lipophilic components, creating a smooth and creamy texture when water is introduced. Thickeners contribute to the body and spreadability of the resulting gel or cream. Emulsifiers 14 and thickeners 16 are used at a concentration of approximately 4% to 10% by weight. Suitable emulsifying and thickening agents include stearic acid, glyceryl stearate, xanthan gum, and various carbomers. Equivalents may include cetearyl alcohol, polyglyceryl esters, or guar gum.

The hydroreactive solid compound include gelling agents 18, which help convert the dissolved tablet into a gel or cream with appropriate viscosity upon hydration. The gelling agents 18 are typically included at 0.5% to 2% by weight. Examples include carbomer, hydroxyethylcellulose, and xanthan gum. Suitable equivalents may include sodium alginate, carrageenan, or methylcellulose. Effervescent agents facilitate tablet disintegration and provide a mild foaming effect when water is added. Sodium bicarbonate is used for its mild alkalinity and ability to promote tablet hardness.

The hydroreactive solid compound include a citric acid 20 to regulate pH and activate the effervescent reaction. Sodium bicarbonate is typically added in the range of 5% to 10% by weight, and citric acid in the range of 1% to 2% by weight. Equivalent effervescent systems may include potassium bicarbonate and tartaric acid.

The hydroreactive solid compound includes binding agents 22 to ensure the mechanical stability of the tablet and help maintain its solid form until use. These are typically included at concentrations of 10% to 20% by weight. Suitable binders include comstarch, arrowroot powder, and kaolin clay. Acceptable equivalents include rice starch, bentonite clay, or microcrystalline cellulose. Surfactants generate lather, helping to lift hair and cleanse the skin during shaving. Surfactants are typically present at levels between 2% and 10% by weight. Examples include sodium lauryl sulfate and sodium cocoyl isethionate (a milder, naturally derived surfactant). Other equivalents may include decyl glucoside, coco betaine, or disodium lauryl sulfosuccinate.

The hydroreactive solid compound includes moisturizing agents 24 to prevent skin dryness and enhance post-shave feel. These are included at concentrations of 2% to 8% by weight. Glycerin is a commonly used humectant, while aloe vera extract, coconut oil, and shea butter provide additional hydration and skin conditioning. Equivalents may include panthenol, allantoin, or squalane. For sensory appeal and aromatherapeutic benefits, essential oils may be added in concentrations ranging from 0.1% to 2% by weight. Suitable oils include lavender, peppermint, and tea tree oil. Menthol may also be added for a cooling effect.

These components may be omitted or replaced with synthetic or hypoallergenic fragrance alternatives, depending on user sensitivity or regulatory preference. If the product is intended for long-term storage or commercial distribution, a preservative may be included in the range of 0.1% to 1% by weight. Suitable preservatives include Optiphen, phenoxyethanol, or natural alternatives such as vitamin E or rosemary extract.

Alluding to the above, a method of manufacture of the aforementioned hydroreactive solid compound is disclosed and generally shown at 30. The hydroreactive shaving tablet 10 is produced via a sequence of heating, blending, molding, and drying steps to achieve a stable, portable form that activates upon hydration. Solid fats (comprising 35% to 55% of the total composition) are melted in a double boiler or microwave-safe container until fully liquefied. Preferred fats include shea butter, cocoa butter, or mango butter. Once the fats are fully melted, emulsifiers (present at 4% to 10% of total weight) such as stearic acid or glyceryl stearate are added and stirred until completely dissolved.

The method further includes step of slowly dispersed gelling agents (at 0.5% to 2%) such as carbomer or xanthan gum into the warm base while stirring continuously to prevent clumping. Binding agents like kaolin clay or cornstarch (at 10% to 20%) are also added at this stage. The method further includes step of blending sodium bicarbonate (5% to 10%) and citric acid (1% to 2%) into the mixture, followed by surfactants (2% to 10%) such as sodium cocoyl isethionate or sodium lauryl sulfate. Mixing continues until a homogenous, semi-solid mass is achieved.

Alluding to the above, moisturizers and conditioners (2% to 8%) like glycerin, aloe vera, or coconut oil are added and blended thoroughly. Optional fragrance oils or cooling agents may be introduced at this stage (0.1% to 2%).

The blended compound is poured or pressed into silicone molds shaped for tablets or pills. Compression may be applied to improve compaction and structural integrity. The filled molds are allowed to cool and solidify at ambient temperature for 6 to 12 hours, or placed in refrigeration to accelerate the process. Upon hardening, tablets are removed from molds and optionally sprayed with essential oils.

The solid shaving tablet is designed for single-use or repeated use depending on size. To activate, the user adds a small amount of water, initiating dissolution and transformation into a creamy or gel-like substance suitable for shaving. The amount of water can be adjusted to achieve the desired viscosity. Minimal water yields a thicker paste, while more water results in a lighter gel.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof.

Therefore, it is intended that the invention not be limited to the particular embodiment disclosed as the best mode contemplated for carrying out this invention. It will be apparent that multiple embodiments of this disclosure may be practiced without some or all of these specific details. In other instances, well-known process operations have not been described in detail in order not to unnecessarily obscure the present embodiments.

1. A hydroreactive solid composition in the form of a disc-shaped molded tablet configured to transform, upon contact with water, into a shaving cream or shaving gel, the hydroreactive solid composition comprising, by weight of the composition:
a solid-fat structural base comprising shea butter and cocoa butter in a combined amount of 35% to 55%;
an emulsifier comprising glyceryl stearate and stearic acid in a combined amount of 4% to 10%;
a gelling agent comprising hydroxyethylcellulose in an amount of 0.5% to 2%;
an effervescent system comprising:
sodium bicarbonate in an amount of 5% to 10%;
citric acid in an amount of 1% to 2%;
a binding agent comprising cornstarch and kaolin clay in a combined amount of 10% to 20%;
a surfactant comprising sodium cocoyl isethionate in an amount of 2% to 10%; and
a moisturizer or conditioner distinct from the solid-fat structural base and comprising glycerin and aloe vera extract, in a combined amount of 2% to about 8%;
wherein the disc-shaped molded tablet comprises a homogeneous solid matrix throughout which the emulsifier, gelling agent, effervescent system, binding agent, surfactant, and moisturizer or conditioner are distributed; and
wherein contact with water causes the sodium bicarbonate and citric acid to react, thereby transforming the homogeneous solid matrix into the shaving cream or shaving gel.

2. (canceled)

3. The hydroreactive solid composition of claim 1, wherein the glyceryl stearate and stearic acid are dissolved in a melted mixture of the shea butter and cocoa butter before the composition is molded and solidified.

4. The hydroreactive solid composition of claim 1, wherein the gelling ·agent further comprises xanthan gum.

5. The hydroreactive solid composition of claim 1, wherein the binding agent further comprises arrowroot powder, and wherein the cornstarch, kaolin clay, and arrowroot powder are present in a combined amount of 10% to 20% by weight of the composition and are distributed throughout the homogeneous solid matrix.

6. The hydroreactive solid composition of claim 1, wherein the disc-shaped molded tablet is a compressed tablet formed by compression of the composition within a mold.

7. The hydroreactive solid composition of claim 1, wherein the moisturizer or conditioner further comprises coconut oil, and wherein the glycerin, aloe vera extract, and coconut oil are present in a combined amount of 2% to 8% by weight of the composition and distributed throughout the homogeneous solid matrix.

8. The hydroreactive solid composition of claim 1, wherein the composition is configured for single-use.

9. The hydroreactive solid composition of claim 1, wherein the composition is configured to be stored in a portable travel container.

10. The hydroreactive solid composition of claim 9, wherein the portable travel container is integrated into a razor handle.

11. The hydroreactive solid composition of claim 1, further comprising peppermint oil, wherein the peppermint oil is present in an amount of 0.1% to 2% by weight.

12. (canceled)

13. The hydroreactive solid composition of claim 1, further comprising menthol.

14. The hydroreactive solid composition of claim 1, further comprising a preservative.

15. The hydroreactive solid composition of claim 14, wherein the preservative comprises phenoxyethanol.

16. The hydroreactive solid composition of claim 15, wherein the phenoxyethanol is present in an amount of about 0.1% to about 1% by weight.

17. (canceled)

18. The hydroreactive solid composition of claim 1, wherein an amount of water contacted with the composition is adjustable to vary a viscosity of the shaving cream or shaving gel, with a lesser amount of water producing a thicker paste and a greater amount of water producing a lighter gel.

19. A single-use hydroreactive shaving composition formed as a disc-shaped molded tablet and configured to transform upon contact with water into a shaving cream or shaving gel, the composition comprising, by weight of the composition:
a solid-fat structural base comprising shea butter and cocoa butter in a combined amount of 35% to 55%;
an emulsifier comprising glyceryl stearate and stearic acid in a combined amount of 4% to 10%;
hydroxyethylcellulose in an amount of 0.5% to 2%;
sodium bicarbonate in an amount of 5% to 10%;
citric acid in an amount of 1% to 2%;
a binding agent comprising cornstarch and kaolin clay in a combined amount of 10% to 20%;
sodium cocoyl isethionate in an amount of 2% to 10%;
a moisturizer or conditioner, distinct from the solid-fat structural base, comprising glycerin and aloe vera extract in a combined amount of 2% to 8%;
peppermint oil in an amount of 0.1% to 2%;
phenoxyethanol in an amount of 0.1% to 1%;
wherein the disc-shaped molded tablet comprises a homogeneous solid matrix throughout which the emulsifier, hydroxyethylcellulose, sodium bicarbonate, citric acid, binding agent, sodium cocoyl isethionate, moisturizer or conditioner, peppermint oil, and phenoxyethanol are distributed; and
wherein contact of the disc-shaped molded tablet with water causes the sodium bicarbonate and citric acid to react and transforms the homogeneous solid matrix into the shaving cream or shaving gel.

20-25. (canceled)
